(19) United States
(12) Patent Application Publication
(10) Pub. No.: US 2026/0015324 A1
(43) Pub. Date: Jan. 15, 2026

(54) Processes for Preparing 5-Bromo-3,4-dimethylpyridin-2-amine and 6-Bromo-7,8-dimethyl-[1,2,4]triazolo[1,5-a]pyridine

(71) Applicant: BRISTOL-MYERS SQUIBB COMPANY, Princeton, NJ (US)

(72) Inventors: Matthew Vincent JOANNOU, New Brunswick, NJ (US); Thomas E. LA CRUZ, Flemington, NJ (US); Nana KIM, Princeton, NJ (US); Kira RAGAZZO, Princeton, NJ (US)

(21) Appl. No.: 18/993,025

(22) PCT Filed: Jul. 12, 2023
(86) PCT No.: PCT/US2023/070008

§ 371 Date: Jan. 10, 2025

Related U.S. Application Data
(60) Provisional application No. 63/388,860, filed on Jul. 13, 2022.

(51) Int. Cl. C07D 213/73; B01J 31/24; C07D 213/74; C07D 471/04

(52) U.S. Cl. CPC C07D 213/73; B01J 31/2409; C07D 213/74; C07D 471/04; B01J 2231/40; B01J 2531/0213; B01J 2531/847

(57) ABSTRACT

Disclosed are processes for preparing 5-bromo-3,4-dimethylpyridin-2-amine and 6-bromo-7,8-dimethyl-[1,2,4]triazolo[1,5-a]pyridine: (I), (VI).

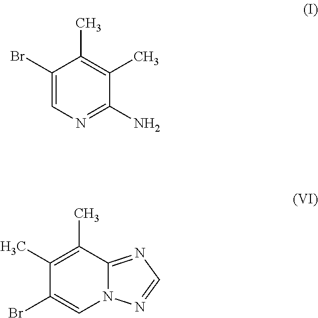

### CROSS REFERENCE

This application claims the benefit of U.S. Provisional Application Ser. No. 63/388,860 filed Jul. 13, 2022, which is incorporated herein in its entirety.

### FIELD OF THE INVENTION

The present invention generally relates to processes for preparing 6-bromo-7,8-dimethyl-[1,2,4]triazolo[1,5-a]pyridine and 5-bromo-3,4-dimethylpyridin-2-amine.

### BACKGROUND OF THE INVENTION

WO 2018/005586 discloses compounds useful in the treatment of inflammatory and autoimmune diseases such as lupus, rheumatoid arthritis, multiple sclerosis, and Sjögren's syndrome, and processes to prepare the compounds and synthesis intermediates. WO 2018/005586 discloses the intermediate 6-bromo-7,8-dimethyl-[1,2,4]triazolo[1,5-a]pyridine (Intermediate F-4) and two processes to prepare the intermediate employing 5-bromo-3,4-dimethylpyridin-2-amine as a starting material.

Desired in the art is an improved process for preparing 6-bromo-7,8-dimethyl-[1,2,4]triazolo[1,5-a]pyridine.

Desired in the art is a process for preparing 6-bromo-7,8-dimethyl-[1,2,4]triazolo[1,5-a]pyridine with a high yield.

Desired in the art is a process for preparing 6-bromo-7,8-dimethyl-[1,2,4]triazolo[1,5-a]pyridine with a high yield and that is adaptable to large scale manufacturing.

Also desired in the art is an improved process for preparing 5-bromo-3,4-dimethylpyridin-2-amine.

Desired in the art is a process for preparing 5-bromo-3,4-dimethylpyridin-2-amine with a high yield.

Desired in the art is a process for preparing 5-bromo-3,4-dimethylpyridin-2-amine with a high yield and that minimizes the formation of side-products.

Desired in the art is a process for preparing 5-bromo-3,4-dimethylpyridin-2-amine with a high yield and that is adaptable to large scale manufacturing.

Desired in the art is a process for preparing 5-bromo-3,4-dimethylpyridin-2-amine with a high yield, that minimizes the formation of side-products, and is adaptable to large scale manufacturing.

Desired in the art is a process for preparing 5-bromo-3,4-dimethylpyridin-2-amine that is adaptable to large scale manufacturing and does not require the use of a palladium catalyst.

Desired in the art is a process for preparing 5-bromo-3,4-dimethylpyridin-2-amine that does not require the use of a palladium catalyst.

Desired in the art is a process for preparing 5-bromo-3,4-dimethylpyridin-2-amine with a high yield, that minimizes the formation of side-products, is adaptable to large scale manufacturing, and does not require the use of a palladium catalyst.

Applicants have discovered a new synthesis process for the preparation of 6-bromo-7,8-dimethyl-[1,2,4]triazolo[1,5-a]pyridine. The process provides 6-bromo-7,8-dimethyl-[1,2,4]triazolo[1,5-a]pyridine in high yield and is adaptable to large scale manufacturing.

Applicants have discovered a new synthesis process for the preparation of 5-bromo-3,4-dimethylpyridin-2-amine. The process provides 5-bromo-3,4-dimethylpyridin-2-amine in high yield, minimizes the formation of side-products, is adaptable to large scale manufacturing, and/or does not employ a palladium catalyst.

### SUMMARY OF THE INVENTION

The present invention provides a synthesis process for making 6-bromo-7,8-dimethyl-[1,2,4]triazolo[1,5-a]pyridine.

The present invention provides intermediates and a process for preparing intermediates useful in the process of preparing 6-bromo-7,8-dimethyl-[1,2,4]triazolo[1,5-a]pyridine.

Additionally, the present invention also provides a synthesis process for making 5-bromo-3,4-dimethylpyridin-2-amine.

Furthermore, the present invention provides a synthesis process for making (E)-N′-(3,5-dibromo-4-methylpyridin-2-yl)-N,N-dimethylformimidamide.

Still furthermore, the present invention provides a synthesis process for making (E)-N′-(5-bromo-3,4-dimethylpyridin-2-yl)-N,N-dimethylformimidamide.

These and other features of the invention will be set forth in expanded form as the disclosure continues.

### BRIEF DESCRIPTION OF THE DRAWINGS

FIG. 1 shows a general reaction schematic for the synthesis of 5-bromo-3,4-dimethylpyridin-2-amine from 2-amino-3,5-dibromo-4-methylpyridine, wherein dmf⋅dma is N,N-dimethylformamide-dimethylacetal.

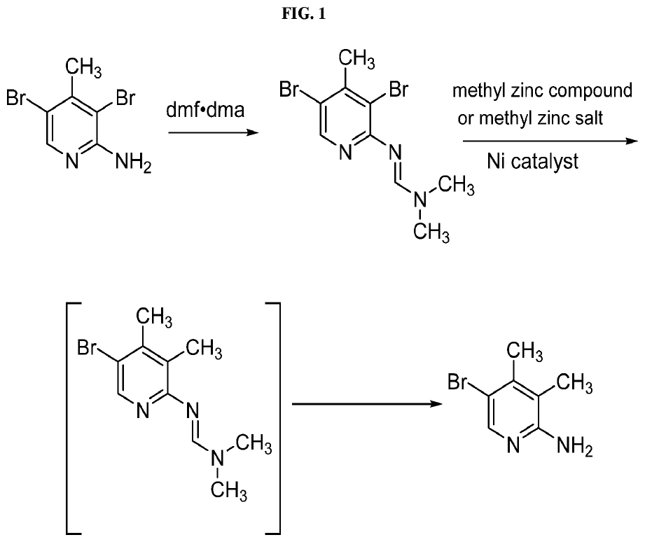

FIG. 2 shows the general reaction schematic for the synthesis of 6-bromo-7,8-dimethyl-[1,2,4]triazolo[1,5-a]pyridine as disclosed in WO 2018/005586.

FIG. 3 shows a general reaction schematic for the synthesis of 6-bromo-7,8-dimethyl-[1,2,4]triazolo[1,5-a]pyridine.

### DETAILED DESCRIPTION

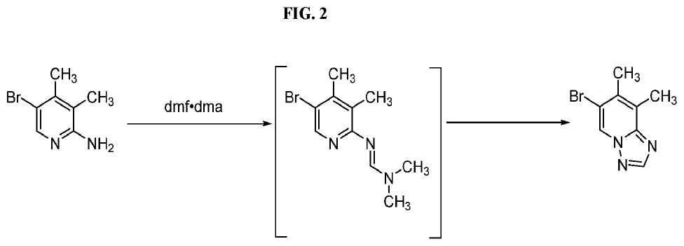

FIG. 2 shows the synthesis of 6-bromo-7,8-dimethyl-[1,2,4]triazolo[1,5-a] pyridine from 5-bromo-3,4-dimethylpyridin-2-amine, as disclosed in WO 2018/005586.

The first aspect of the invention provides a process for preparing a compound of Formula (I):

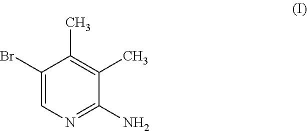

comprising the steps of:
(a) reacting a compound of Formula (II) and a compound of Formula (III):

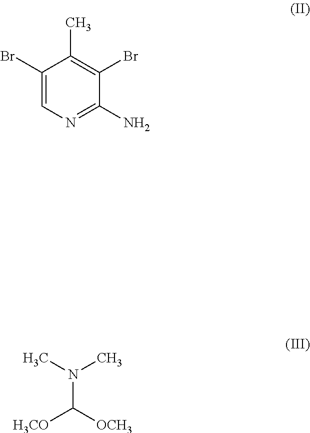

to provide a compound of Formula (IV):

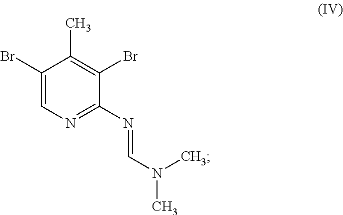

(b) reacting said compound of Formula (IV) with a methyl zinc compound or methyl zinc salt in the presence of a nickel catalyst to provide said compound of Formula (V);

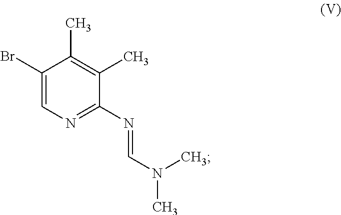

and
(c) hydrolyzing said compound of Formula (V) to provide said compound of Formula (I).

The second aspect of the invention provides a process for preparing a compound of Formula (I):

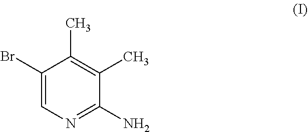

comprising the step of reacting a compound of Formula (IV):

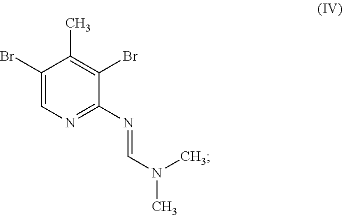

with a methyl zinc compound or methyl zinc salt in the presence of a nickel catalyst to provide said compound of Formula (V);

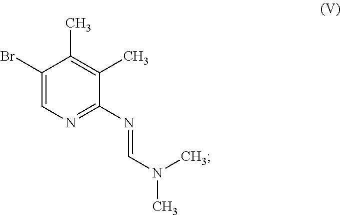

and hydrolyzing said compound of Formula (V) to provide said compound of Formula (I).

The third aspect of the invention provides a process for preparing a compound of Formula (IV) comprising the step of reacting a compound of Formula (II) and a compound of Formula (III):

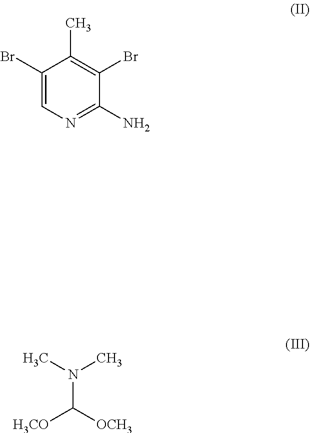

to provide said compound of Formula (IV):

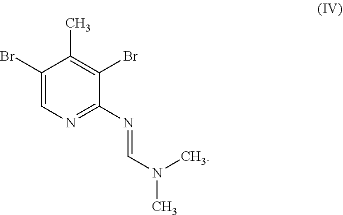

The fourth aspect of the invention provides a compound having the structure of Formula (IV):

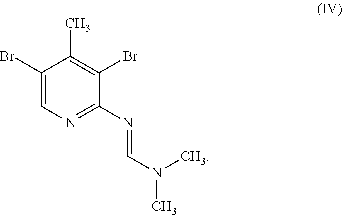

The fifth aspect of the invention provides a process for preparing a compound of Formula (VI):

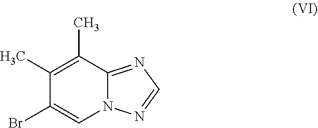

comprising the steps of:
(i) reacting a compound of Formula (IV):

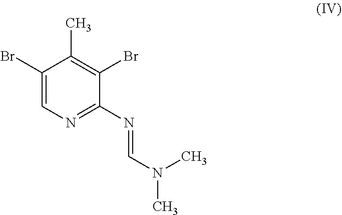

with a methyl zinc compound or methyl zinc salt in the presence of a nickel catalyst to provide said compound of Formula (V):

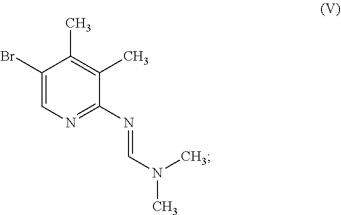

and
(ii) reacting said compound of Formula (V) with hydroxylamine-O-sulfonic acid and dimethylformamide-dimethylacetal to provide said compound of Formula (VI).

The sixth aspect of the invention provides a process for preparing a compound of Formula (VI):

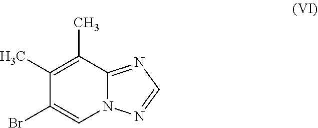

comprising the step of reacting a compound of Formula (V);

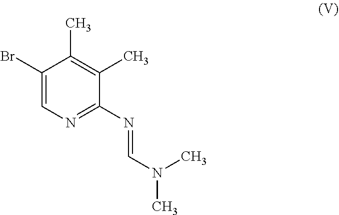

with hydroxylamine-O-sulfonic acid and dimethylformamide-dimethylacetal to provide said compound of Formula (VI).

The seventh aspect of the invention provides a process for preparing a compound of Formula (V):

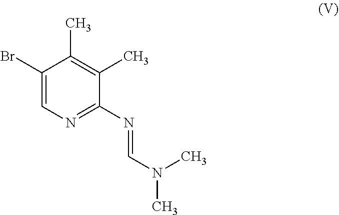

comprising the step of reacting a compound of Formula (IV):

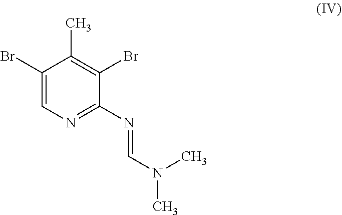

with a methyl zinc compound or methyl zinc salt in the presence of a nickel catalyst to provide said compound of Formula (V).

### PROCESS FOR THE PREPARATION OF 5-BROMO-3,4-DIMETHYLPYRIDIN-2-AMINE

The process for preparing the compound of Formula (I) includes the formation of a carbon-carbon bond by replacing the 3-bromo substituent on the pyridine ring of the starting material, 2-amino-3,5-dibromo-4-methylpyridine, with a methyl group.

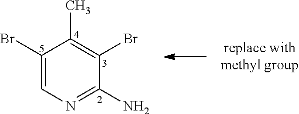

The first step of the process (Step (a) is placing a directing group on the amine group by reacting 2-amino-3,5-dibromo-4-methylpyridine, the compound of Formula (II), with a suitable directing group such as 1,1-dimethoxy-N,N-dimethylmethanamine, to provide (E)-N′-(3,5-dibromo-4-methylpyridin-2-yl)-N,N-dimethylformimidamide, the compound of Formula (IV):

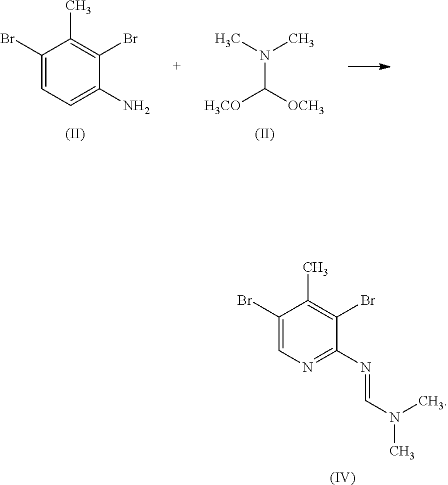

In the second step of the process (Step (b)), the 3-bromo substituent is replaced with a methyl group by reacting the compound of Formula (IV) with a methyl zinc compound or methyl zinc salt in the presence of a nickel catalyst to provide the compound of Formula (V)

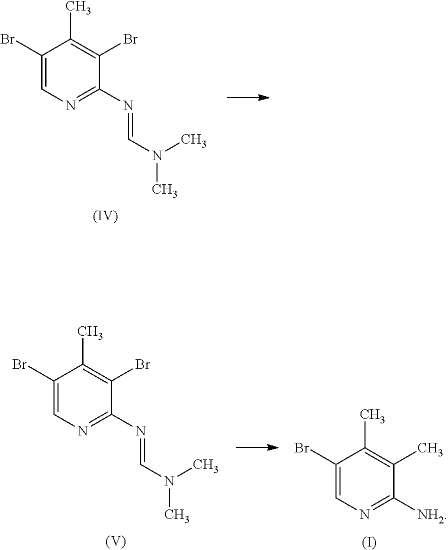

After the replacement of the 3-bromo substituent with the methyl group, the directing group can be removed by hydrolysis in the presence of an acid to regenerate the amine group in the compound of Formula (I). The compound of Formula (V) can be hydrolyzed without isolation to provide the compound of Formula (I); or alternatively, isolated, resolubilized, and then hydrolyzed to provide the compound of Formula (I).

The second step of the process employs a Negishi coupling reaction to replace the 3-bromo group of (E)-N′-(3,5-dibromo-4-methylpyridin-2-yl)-N,N-dimethylformimidamide with a methyl group. Negishi coupling reactions are discussed in Haas et al., ACS Catalysis 2016, 6, 1540-1553; Phapale et al., Chemical Society Reviews 2009, 38, 1598-1607; Jana et al., Chemical Reviews 2011, 111, 1417-1492; Eckert et al., Angew. Chem. Int. Ed. 2021, 60, 12224-12241; Nielson et al., Journal of the American Chemical Society, 2013. 135, 13605-13609; and Tollefson et al., Accounts of Chemical Research 2015, 48, 2344-2353.

### Step (a):

Various synthetic conditions can be employed to prepare the compound of Formula (IV) by reacting the compound of Formula (II) and the compound of Formula (III).

The reaction is sensitive to oxygen. Sparging of the reagents and/or the reaction mixture with an inert gas such as nitrogen or argon may be employed to displace any dissolved oxygen prior to the start of the reaction.

The reaction is sensitive to water as compound of Formula (III) is degraded by water. The water content of the reagents and the reaction mixture should be minimized to suitable water levels, such as, for example, less than 0.2 wt %, less than 0.15 wt %, less than 0.1 wt %, and less than 0.05 wt %.

The reaction between the compound of Formula (II) and the compound of Formula (III) to provide the compound of Formula (IV) can be conducted in various solvents or mixtures thereof. Examples of suitable solvents include, for example, polar aprotic solvents such as dimethyl formamide and N-methyl-2-pyrrolidone; ethereal solvents such tetrahydrofuran, 2-methyl tetrahydrofuran, cyclopentyl methyl ether, and 1,2-dimethoxyethane; alcoholic solvents such as 2-propanol; and other solvents such as toluene.

In one embodiment, the process of Step (a) is conducted in a solvent selected from 2-propanol and 2-methyltetrahydrofuran.

In one embodiment, the process of Step (a) is conducted in a solvent selected from 2-propanol.

In one embodiment, the process of Step (a) is conducted in a solvent selected from 2-methyltetrahydrofuran.

Suitable reaction temperatures for the reaction between the compound of Formula (II) and the compound of Formula (III) include temperatures in the range of from about 70° C. to about 80° C., temperatures in the range of from about 75° C. to about 80° C., and temperatures in the range of from about 78° C. to about 80° C.

The compound of Formula (IV) can be isolated and/or purified by various methods known in the art. Suitable methods include chromatography and crystallization from 2-propanol at approximately 0° C. and collection of the solids by filtration (purity >98%).

### Step (b):

Various synthetic conditions can be employed in Step (b) to prepare the compound of Formula (V).

Suitable nickel catalysts include nickel chloride (NiCl₂) with ligands such as 1,1-bis(diphenylphosphino)methane (dppm), (2,2′-bis(diphenylphosphino)-1,1′-binaphthyl) (binap), 1,3-bis(dicyclohexylphosphino)propane (depp), 1,2-bis(diphenylphosphino)benzene (dppbz), 1,2-bis(dicyclohexylphosphino)ethane (dcpe), 1,1′-bis(diisopropylphosphino)ferrocene (dippf), and triphenylphosphine (PPh₃).

In one embodiment, the process of Step (b) is conducted in the presence of a catalyst selected from (bis(diphenylphosphino)propane)nickel chloride.

Examples of suitable methyl zinc compounds and methyl zinc salts include dimethylzinc and a combination of anhydrous zinc bromide and a Grignard reagent. Suitable Grignard reagents include methyl magnesium chloride, methyl magnesium bromide, and methyl magnesium iodide.

The reaction of compound of Formula (IV) in Step (b) to provide the compound of Formula (V) can be conducted in the presence of various optional synthesis adjuvants, including, for example, alkali metal halides and carboxylates such as LiCl, LiBr, NaBr, lithium acetate, and lithium pivalate; and alkaline earth metal halides and carboxylates such as MgCl₂, MgBr₂, and magnesium pivalate.

The Step (b) reaction is sensitive to oxygen and requires sparging the reagents and/or the reaction mixture with an inert gas such as nitrogen or argon to remove dissolved oxygen prior to the addition of the methyl zinc compound or methyl zinc salt to the reaction mixture.

The Step (b) reaction may be conducted in the presence of low levels of water, such as 0.4 wt % or less. The water content of the reagents and the reaction mixture should be minimized to suitable water levels, such as, for example, less than 0.3 wt %, less than 0.2 wt %, less than 0.1 wt %, and less than 0.05 wt %.

The reaction of compound of Formula (IV) in Step (b) to provide the compound of Formula (V) can be conducted in various solvents or mixtures thereof. Examples of suitable solvents include, but are not limited to, ethereal solvents such as tetrahydrofuran or 2-methyl tetrahydrofuran.

Suitable reaction temperatures for the reaction of compound of Formula (IV) in Step (b) to provide the compound of Formula (V) include temperatures in the range of from about −20° C. to about 25° C., temperatures in the range of from about −10° C. to about 5° C., and temperatures in the range of from about 0° C. to about 5° C.

The compound of Formula (V) can be isolated and/or purified by various methods known in the art. Suitable methods include chromatography and crystallization from acetonitrile, acetone, or tetrahydrofuran, using water as the antisolvent, at approximately 0° C., followed by collection of the solids by filtration. Alternatively, the compound of Formula (V) can be hydrolyzed in Step (c) without isolation from solution.

### Step (c):

Various synthetic conditions can be employed in Step (c) to prepare the compound of Formula (I).

After completion of the methylation reaction, the amidine directing group can be removed under acidic aqueous conditions to provide the compound of Formula (I). Suitable acids include aqueous acids, such as HCl, HBr, HI, and H₂SO₄.

The compound of Formula (I) can be isolated and/or purified by various methods known in the art. Suitable methods include chromatography and crystallization from acetonitrile, using water as the antisolvent, at approximately 0° C., followed by collection of the solids by filtration.

The compound of Formula (I), 5-bromo-3,4-dimethylpyridin-2-amine, is useful as a starting material in the synthesis of 6-bromo-7,8-dimethyl-[1,2,4]triazolo[1,5-a] pyridine, as shown in FIG. 2 and disclosed in WO 2018/005586.

### PROCESS FOR THE PREPARATION OF 6-BROMO-7,8-DIMETHYL-[1,2,4]TRIAZOLO [1,5-A]PYRIDINE

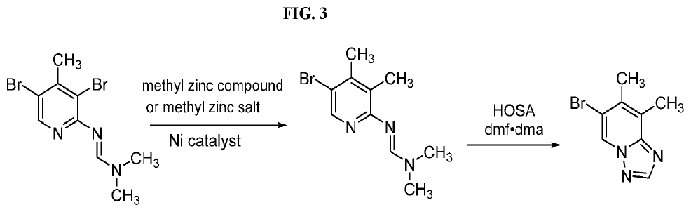

The process for preparing the compound of Formula (VI) from the starting material, (E)-N′-(3,5-dibromo-4-methylpyridin-2-yl)-N,N-dimethylformimidamide, is shown in FIG. 3. The first step of the process, Step (i), includes the formation of a carbon-carbon bond by replacing the 3-bromo substituent on the pyridine ring of (E)-N′-(3,5-dibromo-4-methylpyridin-2-yl)-N,N-dimethylformimidamide, the compound of Formula (IV), with a methyl group:

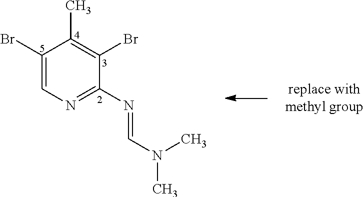

to provide (E)-N′-(5-bromo-3,4-dimethylpyridin-2-yl)-N,N-dimethylformimidamide, the compound of Formula (V). In the second step of the process (Step (ii)), the compound of Formula (V) is reacted to form a triazole ring,

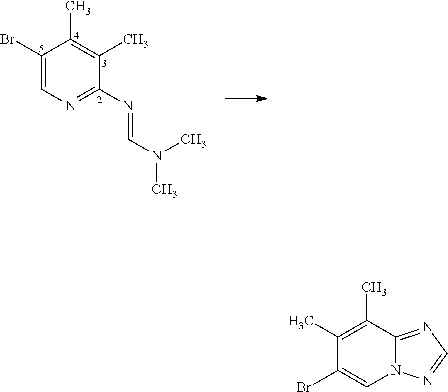

to provide 6-bromo-7,8-dimethyl-[1,2,4]triazolo[1,5-a]pyridine, the compound of Formula (VI).

### Step (i):

Various synthetic conditions can be employed in Step (i), the reaction of the compound of Formula (IV) to replace the 3-bromo substituent with a methyl group to provide the compound of Formula (V).

Suitable nickel catalysts include nickel chloride (NiCl₂) with ligands such as 1,1-bis(diphenylphosphino)methane (dppm), (2,2′-bis(diphenylphosphino)-1,1′-binaphthyl) (binap), 1,3-bis(dicyclohexylphosphino)propane (depp), 1,2-bis(diphenylphosphino)benzene (dppbz), 1,2-bis(dicyclohexylphosphino)ethane (dcpe), 1,1′-bis(diisopropylphosphino)ferrocene (dippf), and triphenylphosphine (PPh₃).

In one embodiment, the process of Step (i) is conducted in the presence of a catalyst selected from (bis(diphenylphosphino)propane)nickel chloride.

Examples of suitable methyl zinc compounds and methyl zinc salts include dimethylzinc and a combination of anhydrous zinc bromide and a Grignard reagent. Suitable Grignard reagents include methyl magnesium chloride, methyl magnesium bromide, and methyl magnesium iodide.

The reaction of compound of Formula (IV) in Step (i) to provide the compound of Formula (V) can be conducted in the presence of various optional synthesis adjuvants, including, for example, alkali metal halides and carboxylates such as LiCl, LiBr, NaBr, lithium acetate, and lithium pivalate; and alkaline earth metal halides and carboxylates such as MgCl₂, MgBr₂, and magnesium pivalate.

The Step (i) reaction is sensitive to oxygen and requires sparging the reagents and/or the reaction mixture with an inert gas such as nitrogen or argon to remove dissolved oxygen prior to the addition of dimethyl zinc to the reaction mixture.

The Step (i) reaction may be conducted in the presence of low levels of water, such 0.4 wt % or less. The water content of the reagents and the reaction mixture should be minimized to suitable water levels, such as, for example, less than 0.3 wt %, less than 0.2 wt %, less than 0.1 wt %, and less than 0.05 wt %.

The reaction of compound of Formula (IV) in Step (i) to provide the compound of Formula (V) can be conducted in various solvents or mixtures thereof. Examples of suitable solvents include, but are not limited to ethereal solvents such as tetrahydrofuran or 2-methyl tetrahydrofuran.

Suitable reaction temperatures for the reaction of compound of Formula (IV) in Step (i) to provide the compound of Formula (V) include temperatures in the range of from about −20° C. to about 25° C., temperatures in the range of from about −15° C. to about 10° C., temperatures in the range of from about −15° C. to about 5° C., and temperatures in the range of from about −5° C. to about 5° C.

The compound of Formula (V) can be isolated and/or purified by various methods. Suitable methods include chromatography and crystallization from acetonitrile, acetone, or tetrahydrofuran using water as the antisolvent, at approximately 0° C., followed by collection of the solids by filtration.

### Step (ii):

Various synthetic conditions can be employed in Step (ii), the reaction of the compound of Formula (V) to form a triazole ring to provide the compound of Formula (VI).

The reaction can be conducted in the present of dimethylformamide-dimethylacetal (DMF-DMA) and a suitable acid, such as hydroxylamine-O-sulfonic acid. Suitable levels of DMF-DMA include amounts in the range of 0.5 to 1.3 equivalents, in the range of 0.7 to 1.2 equivalents, and in the range of 0.8 to 1.1 equivalents. Suitable levels of hydroxylamine-O-sulfonic acid include amounts in the range of 1,0 to 2.0 equivalents, in the range of 1.1 to 1.7 equivalent, and in the range of 1.4 to 1.6 equivalents.

The reaction of the compound of Formula (V) to provide the compound of Formula (VI) can be conducted in various solvents or mixtures thereof. Examples of suitable solvents include, for example, polar aprotic solvents such as dimethyl formamide and N-methyl-2-pyrrolidone; ethereal solvents such tetrahydrofuran, 2-methyl tetrahydrofuran, cyclopentyl methyl ether, and 1,2-dimethoxyethane; alcoholic solvents such as 2-propanol; and other solvents such as toluene.

In one embodiment, the process of Step (ii) is conducted in a solvent selected from 2-propanol and 2-methyltetrahydrofuran.

In one embodiment, the process of Step (ii) is conducted in a solvent selected from 2-propanol.

In one embodiment, the process of Step (ii) is conducted in a solvent selected from 2-methyltetrahydrofuran.

Suitable reaction temperatures for the reaction between the compound of Formula (V) to form the compound of Formula (VI) include temperatures in the range of from about 40° C. to about 70° C., temperatures in the range of from about 40° C. to about 60° C., and temperatures in the range of from about 45° C. to about 55° C.

The compound of Formula (VI) can be isolated and/or purified by various methods known in the art. Suitable methods include chromatography and crystallization from 2-propanol at approximately 0° C. and collection by filtration.

The present invention may be embodied in other specific forms without departing from the spirit or essential attributes thereof. This invention encompasses all combinations of the aspects and/or embodiments of the invention noted herein. It is understood that any and all embodiments of the present invention may be taken in conjunction with any other embodiment or embodiments to describe additional embodiments. It is also to be understood that each individual element of the embodiments is meant to be combined with any and all other elements from any embodiment to describe an additional embodiment.

### Definitions

The features and advantages of the invention may be more readily understood by those of ordinary skill in the art upon reading the following detailed description. It is to be appreciated that certain features of the invention that are, for clarity reasons, described above and below in the context of separate embodiments, may also be combined to form a single embodiment. Conversely, various features of the invention that are, for brevity reasons, described in the context of a single embodiment, may also be combined so as to form sub-combinations thereof. Embodiments identified herein as exemplary or preferred are intended to be illustrative and not limiting.

Unless specifically stated otherwise herein, references made in the singular may also include the plural. For example, “a” and “an” may refer to either one, or one or more.

Unless otherwise indicated, any atom with unsatisfied valences is assumed to have hydrogen atoms sufficient to satisfy the valences.

The definitions set forth herein take precedence over definitions set forth in any patent, patent application, and/or patent application publication incorporated herein by reference.

Listed below are definitions of various terms used to describe the present invention. These definitions apply to the terms as they are used throughout the specification (unless they are otherwise limited in specific instances) either individually or as part of a larger group.

The compounds of Formulas (I), (II), (III), (IV), (V), and (VI) can be provided as amorphous solids or crystalline solids. Lyophilization can be employed to provide the compounds as a solid.

In addition, compounds of Formulas (I), (II), (III), (IV), (V), and (VI), subsequent to their preparation, can be isolated and purified to obtain a composition containing an amount by weight equal to or greater than 93% of a compound of Formulas (I), (II), (III), (IV), (V), and (VI), (“substantially pure”), which is then used as described herein. Such “substantially pure” compounds of Formulas (I), (II), (III), (IV), (V), and (VI) are also contemplated herein as part of the present invention.

“Stable compound” and “stable structure” are meant to indicate a compound that is sufficiently robust to survive isolation to a useful degree of purity from a reaction mixture.

The compounds of the present invention are intended to include all isotopes of atoms occurring in the present compounds. Isotopes include those atoms having the same atomic number but different mass numbers. By way of general example and without limitation, isotopes of hydrogen include deuterium (D) and tritium (T). Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed. For example, methyl (—CH₃) also includes deuterated methyl groups such as —CD₃.

### EXAMPLES

The invention is further defined in the following Example. It should be understood that the Example is given by way of illustration only. From the above discussion and the Example, one skilled in the art can ascertain the essential characteristics of the invention, and without departing from the spirit and scope thereof, can make various changes and modifications to adapt the invention to various uses and conditions. As a result, the invention is not limited by the illustrative examples set forth herein below, but rather is defined by the claims appended hereto.

ABBREVIATIONS

| | |
|---|---|
| anhyd. | anhydrous |
| aq. | aqueous |
| DMF | dimethylformamide |
| dmf•dma | N,N-dimethylformamide-dimethylacetal |
| dppp | 1,3-bis(diphenylphosphino)propane |
| (dppp)NiCl₂ | [1,3-bis(diphenylphosphino)propane]dichloronickel(II) |
| h, hr or hrs | hour(s) |
| IPA | isopropyl alcohol |
| HPLC | high performance liquid chromatography |
| HOSA | hydroxylamine-O-sulfonic acid |
| iPrOH | isopropanol |
| LC | liquid chromatography |
| LCMS | liquid chromatography mass spectrometry |
| M | molar |
| mM | millimolar |
| Me | methyl |
| MeCN | acetonitrile |
| MeMgCl | methyl magnesium chloride |
| 2-MeTHF | 2-methyl tetrahydrofuran |
| MHz | megahertz |
| min. | minute(s) |
| mins | minute(s) |
| MS | mass spectrometry |
| NH₂OSO₃H | hydroxylamine-O-sulfonic acid |
| nM | nanomolar |
| Ret Time or Rt | retention time |
| sat. | saturated |
| SFC | supercritical fluid chromatography |
| THF | tetrahydrofuran |
| wt % | weight % |
| ZnMe₂ | dimethyl zinc |

### SYNTHESIS OF 5-BROMO-3,4-DIMETHYLPYRIDIN-2-AMINE USING NI CATALYST

### Step (a): Preparation of (E)-N′-(3,5-dibromo-4-methylpyridin-2-yl)-N,N-dimethylformimidamide

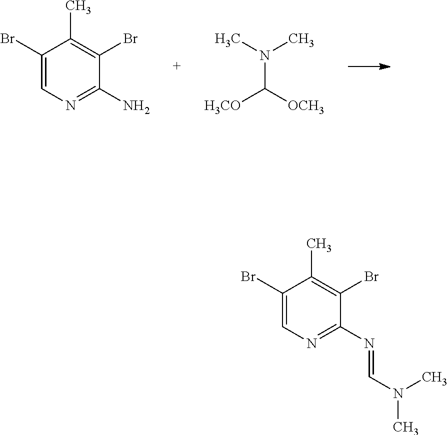

A 250-mL reactor was charged with 2-propanol (150 mL, 0.500 M, 7.50 L/kg) and 2-amino-3,5-dibromo-4-methylpyridine (20.0 g, 75.2 mmol). The reaction mixture was sparged with N₂ with agitation for 15 minutes at ambient temperature. N,N-dimethylformamide-dimethylacetal (13.0 mL, 97.8 mmol, 1.3 equiv.) was added to the reaction mixture under N₂ via a syringe. The reactor jacket was heated to 80° C. and the reaction mixture was aged for 6 hours. The reaction mixture was sampled at 80° C. under a flow of nitrogen pressure and analyzed via UHPLC-MS (ultra-high performance liquid chromatography-mass spectrometry) wherein all starting material was observed to have been consumed. The sample aliquot was removed from the reaction mixture at 80° C. under a flow of N₂ and diluted in MeCN. After completion of the reaction, the contents of the reactor were cooled to 0° C. over 2 hours and the resulting slurry was filtered. The solids were collected, transferred to a bottle, and dried in a vacuum oven at 50° C. for 24 hours to afford (E)-N′-(3,5-dibromo-4-methylpyridin-2-yl)-N,N-dimethylformimidamide as a solid in >98% yield (24.0 g). ¹H NMR (CDCl₃, 500 MHz, 23° C.): δ 8.34 (s, 1H), 8.16 (s, 1H), 3.15 (s, 3H), 3.10 (s, 3H), 2.56 (s, 3H). Trace amounts of water and isopropanol were detected in the product.

### Steps (b) and (c): Preparation of 5-bromo-3,4-dimethylpyridin-2-amine

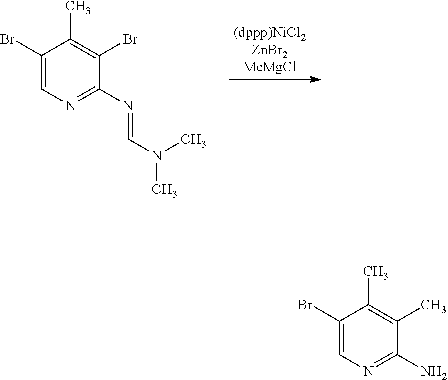

A 5 L reactor was charged with (E)-N′-(3,5-dibromo-4-methylpyridin-2-yl)-N,N-dimethylformimidamide (125 g, 389.4 mmol), (dppp)NiCl₂ (10.5 g, 19.47 mmol), and ZnBr₂ (87.7 g, 389.4 mmol) under N₂ flow. 2-MeTHF (2.5 L, 20 L/kg) was added to the reactor. As the contents of the reactor were cooled to 0-1° C. (internal temperature), the entire solution was sub-surface sparged with N₂ for 30 minutes. A 2.8 M solution of MeMgCl (278 mL, titrated with sec-butyl alcohol using 1,10-phenanthroline as an indicator before use) was added to the reactor via a syringe over 30 minutes while the internal temperature of the reaction mixture was maintained at less than 4° C. The reaction mixture was aged between 0-1° C. for 6 hours. The reaction mixture was sampled under a flow of nitrogen pressure and analyzed by UHPLC-MS and the reaction was confirmed to be complete with no starting material present (98 AP product, 2 AP bis-methylation; AP=area percent). The reactor was then charged with aqueous 3 M HCl (10.0 equiv., 1.3 L) over 30 minutes. The batch was heated to 65° C. with agitation for 18 hours. The batch was then cooled to 20° C. and sampled under a flow nitrogen pressure and analyzed by UHPLC-MS to afford S-bromo-3,4-dimethylpyridin-2-amine in 95 AP. Next, 25% aqueous NHAOH (˜10.0 equiv.) was charged into the reactor with agitation until the pH was adjusted to 5.0-5.5. Agitation was halted and the layers were allowed to separate for 15 minutes. The bottom aqueous layer was drained from the reactor and discarded to waste. Next, 25% aqueous NH₄OH (5.0 equiv., 145 mL) was added to the reactor and the batch was heated to 50° C. with agitation for 18 hours. The batch was cooled to 20° C. and agitation halted. The layers were allowed to settle for 15 minutes and the bottom aqueous layer was drained from the reactor and discarded to waste. The batch was distilled to 10 L/kg at 60° C. jacket temperature and ˜100 mbar. Acetonitrile (10 L/kg, 1.25 L) was charged to the reactor and the batch distilled down to 4 L/kg. Acetonitrile (10 L/kg, 1.25 L) was charged to the reactor and the batch distilled down to 4 L/kg. At ˜50° C. internal temperature, water (10 L/kg, 1.25 L) was added to the reactor over 15 minutes. The batch was cooled to 10° C. over 2 hours to afford a slurry, which was aged for 12 hours. The solid was collected on a polypropylene filter funnel with a 40 micron polyethylene fritted disc. The reactor was rinsed with 1:2 MeCN:water (2 L/kg total, 250 mL) and the rinse was passed through the filter cake. The solid was then dried in vacuo at 50-55° C. to afford 5-bromo-3,4-dimethylpyridin-2-amine in 83% yield (64.9 g, 95% purity). ¹H NMR (CDCl₃, 500 MHz, 23° C.): δ 8.02 (s, 1H), 4.34 (br s, 2H), 2.34 (s, 3H), 2.11 (s, 3H).

### Comparison of Ni and Pd Catalysts in the Preparation of (E)-N′-(5-bromo˜3,4-dimethylpyridin-2-yl)-N,N-dimethylformimidamide

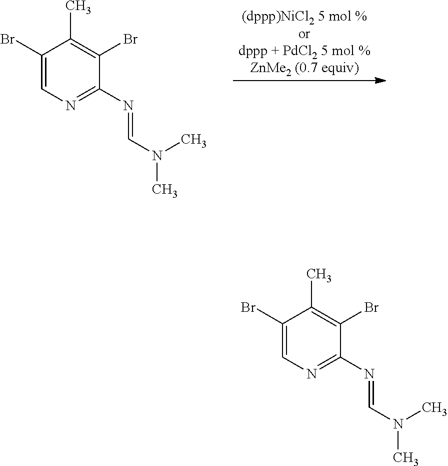

A 40-mL scintillation vial was charged with (E)-N′-(3,5-dibromo-4-methylpyridin-2-yl)-N,N-dimethylformimidamide (250 mg, 0.779 mmol) and either (i) (dppp)NiCl₂ (21.1 mg, 0.0390 mmol) or (ii) PdCl₂ (7.0 mg, 0.0390 mmol) and dppp (16.1 mg, 0.0390 mmol). A magnetic stir-bar was added to the vial. Tetrahydrofuran (6.0 mL, 24 L/kg) was added to the vial via syringe. The solution was sparged with N₂ with rapid stirring for 15 minutes. The reaction mixture was cooled to 0-1° C. (aluminum reaction block immersed in ice) and ZnMe₂ (0.83 mL, 0.545 mmol) was added to the reaction mixture as a 0.66 M solution in hexane under N₂ via syringe. The reaction mixture was allowed to warm to ambient temperature and stirred for 18 hours. An aliquot was removed from the reaction mixture and analyzed via UPLC-MS. AP=area percent of chromatogram peak integration.

TABLE 1
Comparison of Synthesis of (E)-N′-(5-bromo-3,4-dimethylpyridin-2-yl)-N,N-
dimethylformimidamide with Ni or Pd catalysts

| | | | Pd |
|---|---|---|---|
| | | Ni catalyst | catalyst |
| Compound | Structure | (yield) | (yield) |
| V | 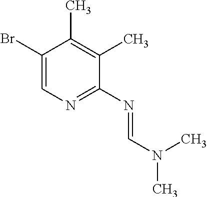 | 91.7 AP | 28.2 AP |
| IV | 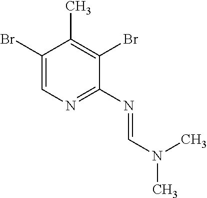 | <0.05 AP | 52.5 AP |
| | Starting material | | |
| B | 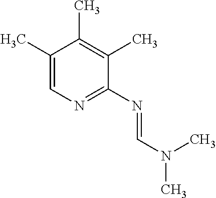 | 8.0 AP | 9.1 AP |
| C | 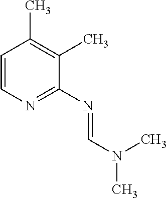 | 0.3 AP | 0.4 AP |
| D | 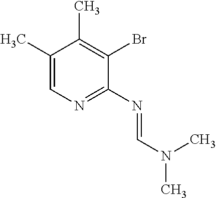 | <0.05 AP | 9.3 AP |
| E | 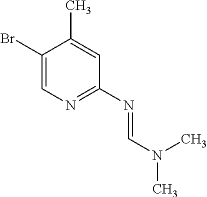 | <0.05 AP | 0.55 AP |

According to the above comparison of the synthesis of (E)-N′-(5-bromo-3,4-dimethylpyridin-2-yl)-N,N-dimethylformimidamide with Ni or Pd catalysts, the results in Table 1 show that the process using the Ni catalyst produced (E)-N′-(5-bromo-3,4-dimethylpyridin-2-yl)-N,N-dimethylformimidamide in higher yield than the process using the Pd catalyst (Compound of Formula (V): 91.7 AP versus 28.2 AP, respectively) with complete consumption of the starting material (Compound of Formula (IV): <0.05 AP versus 52.5 AP, respectively). Additionally, in this experiment, the results in Table 1 show that the use of the Ni catalyst led to the formation of fewer and smaller quantities of side products than the process using the Pd catalyst (Compounds B to E: <8.5 AP versus approximately 19.3 AP, respectively).

Applicants have discovered a new synthesis process for the preparation of 5-bromo-3,4-dimethylpyridin-2-amine. The new synthesis process minimizes the production of side products. The new synthesis process is adaptable to large scale manufacturing.

### SYNTHESIS OF (E)-N′-(5-BROMO-3,4-DIMETHYLPYRIDIN-2-YL)-N,N-DIMETHYL FORMIMIDAMIDE

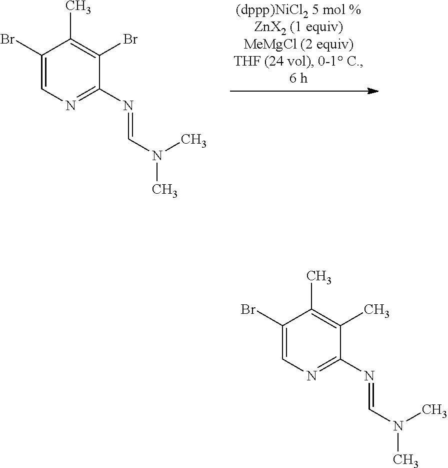

A 250-mL reactor was charged with (E)-N′-(3,5-dibromo-4-methylpyridin-2-yl)-N,N-dimethylformimidamide (3.00 g, 9.35 mmol, limiting reagent) and (dppp)NiCl₂ (261 mg, 0.467 mmol) under N₂ flow. Anhydrous THF (72 mL, 0.13 M) was then charged via syringe under N₂ followed by sparged zinc (2-ethylhexanoate) (2.97 mL, 9.35 mmol). The reaction mixture was cooled to 0-1° C. (internal temperature) after which time a 4.1 M solution of MeMgCl (4.6 mL, titrated before use) was added to the reactor via syringe over 15 minutes, while maintaining the internal temperature of the reaction mixture to less than 4° C.). The color of the reaction mixture changed from slight orange to bright yellow to dark red over the course of the addition. The reaction mixture was aged between 0-1° C. for 6 hours. IPC confirmed complete consumption of the starting material (95 AP product, 5 AP bis-methylation). The reactor was charged with a saturated aqueous solution of NH₄Cl (˜100 mL) and the mixture was agitated for 1 minute. After allowing the layers to separate, the aqueous layer was discarded and the organic layer was washed twice with brine. Final organic layer mass: (62.3 g, in-process yield: 92.1%). Concentrated organic layer in vacuo to afford the product as a yellow solid. ¹H NMR (CDCl₃, 500 MHz, 23° C.): δ 8.28 (s, 1H), 8.14 (s, 1H), 3.08 (br s, 6H), 2.36 (s, 3H), 2.34 (s, 3H).

### SYNTHESIS OF 6-BROMO-7,8-DIMETHYL-[1,2,4]TRIAZOLO [1,5-A]PYRIDINE

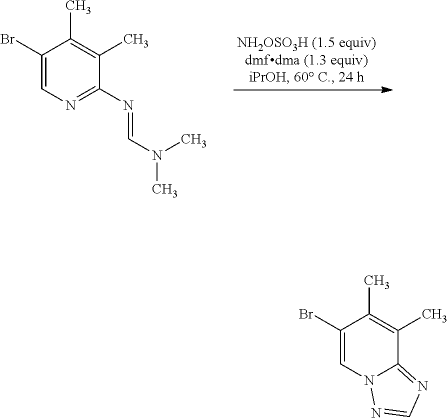

A 40-mL scintillation vial was charged with (E)-N′-(5-bromo-3,4-dimethylpyridin-2-yl)-N,N-dimethylformimidamide (2.87 g, 11.2 mmol, crude from reaction), hydroxylamine-O-sulfonic acid (2.00 g, 16.8 mmol), and a magnetic stir-bar. The vial was purged with N₂ for 10 minutes and then charged with 2-propanol (25.8 mL) and dimethylformamide-dimethylacetal (2.04 mL, 14.6 mmol). The vial was sealed and then heated to 60° C. and agitated for 18 hours. Initially, a thick slurry forms which gives way to a near homogeneous solution at 60° C. IPC confirmed consumption of starting material (in-process yield: 81%). The reaction mixture was transferred to a 250 mL flask and then charged with water over ˜2 hours (37 mL, 13 L/kg) and then cooled to 30° C. over 2 hours. A 2.26 M solution of Na₂CO₃ in water was then charged to the flask and the pH adjusted to 4-5. The flask is then cooled to 0-5° C. over ˜2 hours and the batch aged for 2-3 hours before filtration. The slurry is filtered and the wet cake washed with 2-propanol (˜1 mL) and water (˜5 mL). The solid was then dried in vacuo at 50° C. for 24 hours. ¹H NMR (CDCl₃, 300 MHz, 23° C.): δ 8.66 (s, 1H), 8.24 (s, 1H), 2.65 (s, 6H), 2.47 (s, 6H).

### SYNTHESIS OF (E)-N′-(5-BROMO-3,4-DIMETHYLPYRIDIN-2-YL)-N,N-DIMETHYLFORMIMIDAMIDE

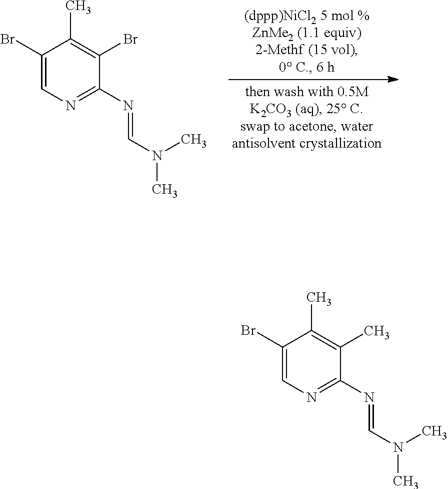

A 1-L reactor was charged with (E)-N′-(3,5-dibromo-4-methylpyridin-2-yl)-N,N-dimethylformimidamide (30.0 g, 93.5 mmol, limiting reagent) and (dppp)NiCl₂ (2.47 g, 4.68 mmol) under N₂ flow. Anhydrous 2-methyltetrahydrofuran (450 mL, 15 L/kg, 0.21 M) was then charged via syringe under N₂. The reaction mixture was cooled to 0° C. (internal temperature) after which time a 1.0 M solution of ZnMe₂ in heptane (103.0 mL, titrated before use) was added to the reactor via syringe over 15 minutes, while maintaining the internal temperature of the reaction mixture below 5° C.). The reaction color changed from slight orange to bright yellow to dark red over the course of the addition. The reaction mixture was aged at 0° C. for 6 hours. IPC confirmed complete consumption of starting material. The reactor was charged with aqueous 0.5 M K₂CO₃ solution (300 mL, 1.5 equiv.) and the mixture agitated for 15 hours. After allowing the layers to separate, the aqueous layer was discarded and the organic layer was washed with water (300 mL) and polish filtered (Whatman). The batch was solvent swapped into acetone with a constant volume distillation at 70 mL using 448 mL of acetone at 300 mbar. The stream bad a homogeneous orange color at the end of distillation. For crystallization, 84 mL of water was charged over 3 hours, followed by 196 mL of water charged over 2 hours. Spontaneous nucleation was observed 36.4 mL into the first water addition. The crystallization slurry was aged overnight. The batch was filtered and rinsed with a single wash of 56 mL water and 14 mL acetone. There was 5% yield loss to mother liquor and 0.3% yield loss to cake wash. The batch was dried in a vacuum oven at 45° C. and 23 mm Hg under nitrogen bleed for 12 hr. IPC for end of drying was <1.0 wt %. Isolation yield was 87.6%. ¹H NMR (CDCl₃, 500 MHz, 23° C.): δ 8.28 (s, 1H), 8.14 (s, 1H), 3.08 (br s, 6H), 2.36 (s, 3H), 2.34 (s, 3H).

### SYNTHESIS OF (E)-N′-(5-BROMO-3,4-DIMETHYLPYRIDIN-2-YL)-N,N-DIMETHYLFORMIMIDAMIDE

This procedure used ZnBr₂ and MeMgCl for in situ generation of ZnMe₂.

A 250-mL reactor was charged with (E)-N′-(3,5-dibromo-4-methylpyridin-2-yl)-N,N-dimethylformimidamide (5.0 g, 15.6 mmol, limiting reagent), (dppp)NiCl₂ (411 mg, 0.778 mmol), and ZnBr₂ (3.51 g, 15.6 mmol) under N₂ flow. Anhydrous 2-methyl tetrahydrofuran (75 mL, 15 L/kg, 0.21 M) was then charged via syringe under N₂. The reaction mixture was cooled to 0° C. (internal temperature) after which time a 3.8 M solution of MeMgCl in THF (8.2 mL, titrated before use) was added to the reactor via syringe over 15 minutes, while maintaining the internal temperature of the reaction mixture below 5° C.). The reaction color changed from slight orange to bright yellow to dark red over the course of the addition. The reaction mixture was aged at 0° C. for 3 hours. IPC confirmed complete consumption of starting material. The reactor was charged with aqueous 0.5 M K₂CO₃ solution (100 mL, 3.5 equiv.), warmed to 25° C. and the mixture agitated for 1 hour. The biphasic mixture was filtered (Whatman) and washed twice with 10 mL THF. After allowing the layers to separate, the aqueous layer was discarded, and the organic layer was washed with water (110 mL). The final organic layer mass: (93.3 g, in-process yield: 92.0%).

### SYNTHESIS OF (E)-N′-(5-BROMO-3,4-DIMETHYLPYRIDIN-2-YL)-N,N-DIMETHYLFORMIMIDAMIDE

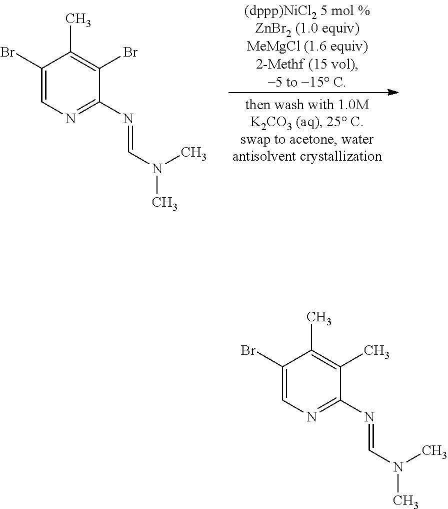

Reactor 1 was charged with anhydrous 2-methyl tetrahydrofuran (15 L/kg) followed by ZnBr₂ (10.6 kg, 1.0 equiv.) at 20° C. The reaction mixture was stirred until the ZnBr₂ was completely dissolved. (E)-N′-(3,5-dibromo-4-methylpyridin-2-yl)-N,N-dimethylformimidamide (15.2 kg, limiting reagent) was charged. The reaction mixture was cooled to −13° C. The reaction mixture was sub-surface sparged with N₂ for 30 minutes. Next, (dppp)NiCl₂ (1.26 kg, 0.05 equiv.) was charged, and the resulting orange-red thin slurry was sub-surface sparged with N₂ for 30 minutes. A solution of MeMgCl in THF (26.0 kg, 3.0 M, titrated before use) was added to the reactor over 3 hours, while the internal temperature was maintained in the range of from −5 to −15° C. The reaction color changed from slight orange to bright yellow to dark red over the course of the addition. The reaction mixture was aged at −5 to −15° C. for 14 hours. IPC confirmed complete consumption of starting material. Next, 1.0 M K₂CO₃ solution (3.0 equiv.) was prepared in Reactor 2 at 5° C., and mixed with diatomite (15 kg). The mixture in Reactor 1 was transferred to Reactor 2 while maintaining the transfer temperature at −5 to −15° C. After transfer, temperature was increased to 16° C. A biphasic mixture was centrifuge-filtered, and transferred to Reactor 1. Agitation was halted and the layers were allowed to separate for 30 minutes. The bottom aqueous layer was discarded, and the organic layer was washed with 5% NaCl solution (10 vol). The aqueous layer was discarded and the organic layer was concentrated to 2˜3 vol (T≤45° C., P<−0.06 MPa). Then, 6 vol acetone was charged, and the mixture was concentrated to 3 vol. At 20° C., the controlled antisolvent addition of water (12 vol) over 3 hours afforded a slurry, which was aged for 2 hours. The solid was collected by filtration, and the reactor was rinsed with a solution of 1.34 vol water and 0.66 vol acetone. The rinse was passed through the filter cake. The wet cake was deliquored and the batch transferred to a Nutsche filter and dried under nitrogen at 36° C. for 15 hours. The batch was then sampled for KF (KF NMT 0.5 wt %), acetone residual (NMT 0.5 wt %). The product was isolated in 81% yield (97.4% LCAP, 97.7 wt %).

### SYNTHESIS OF 6-BROMO-7,8-DIMETHYL-[1,2,4]TRIAZOLO [1,5-A]PYRIDINE

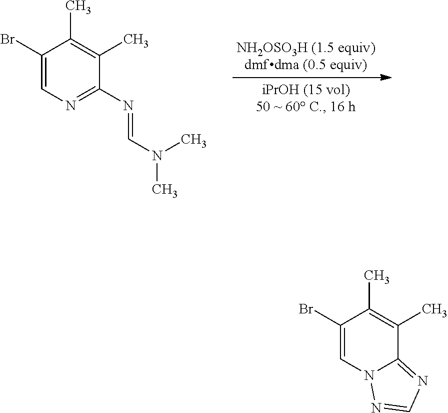

2-Propanol (15 L/kg) was added into the Reactor 1, and the temperature was adjusted to 22° C. Next, 1.0 eq (E)-N′-(5-bromo-3,4-dimethylpyridin-2-yl)-N,N-dimethylformimidamide (129.2 kg, limiting reagent) and 0.5 eq. DMF-DMA were charged in sequence. The temperature was raised to 50° C. and HOSA was added in 6 portions. The mixture was aged for 19.5 h. Upon completion of the reaction completion (IPC), the mixture was distillated to 3 vol at 45° C. under vacuum, and temperature was adjusted to 20° C. DCM (S vol) and water (8 vol) were charged, and Na₂CO₃ solution (12 wt %) was charged to adjust pH to 6 at 20° C. Layers were separated and the bottom organic layer was collected. The top aqueous layer was discarded. An aqueous HCl solution (0.5 M, 2 L/kg) was prepared, and organic mixture was washed twice with 0.5 M HCl (2 L/kg) twice, followed by water (4 L/kg) wash. The separated organic mixture was polish filtered and concentrated to 2 L/kg at 16° C. under vacuum, and 2-propanol (2 L/kg) was charged and concentrated to 2 vol. The residual DCM level in the mixture was ≤1 wt %. The temperature was adjusted to 23° C., and water (8 L/kg) was charged. The batch was cooled to 0° C. and aged for 8 hours. The slurry was filtered, and the wet cake washed with 3:1 water: 2-propanol (1 L/kg total). The batch was dried under vacuum at 63° C. for 32 hours (KF NMT 0.5 wt %, IPC residual NMT 0.5 wt %). 6-Bromo-7,8-dimethyl-[1,2,4]triazolo[1,5-a]pyridine, was isolated at 73% yield (99.3% LCAP, 99.8 wt %).

What is claimed is:

1. A process for preparing a compound of Formula (I): 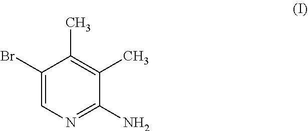
comprising the steps of:
(a) reacting a compound of Formula (II) and a compound of Formula (III): 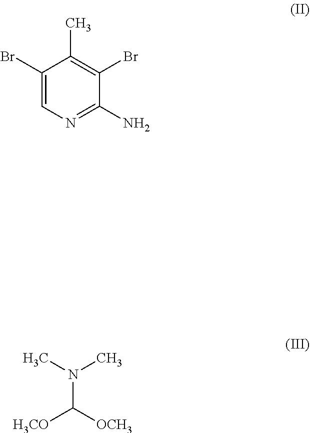
to provide a compound of Formula (IV): 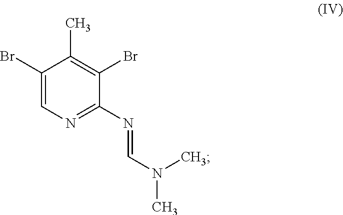
(b) reacting said compound of Formula (IV) with a methyl zinc compound or methyl zinc salt in the presence of a nickel catalyst to provide said compound of Formula (V); 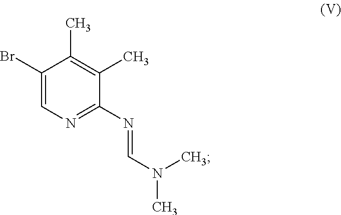
and
(c) hydrolyzing said compound of Formula (V) to provide said compound of Formula (I).

2. The process according to claim 1 wherein said nickel catalyst is NiCl₂ with a ligand selected from 1,1-bis(diphenylphosphino)methane, (2,2′-bis(diphenylphosphino)-1,1′-binaphthyl), 1,3-bis(dicyclohexylphosphino)propane, triphenylphosphine, 1,2-bis(diphenylphosphino)benzene, 1,2-bis(dicyclohexylphosphino)ethane, and 1,1′-bis(diisopropylphosphino)ferrocene.

3. The process according to claim 1 wherein said nickel catalyst is bis(diphenylphosphino)propane)nickel chloride.

4. The process according to claim 1 wherein said methyl zinc compound or methyl zinc salt is dimethylzinc or a combination of anhydrous zinc bromide and Grignard reagent.

5. A process for preparing a compound of Formula (I): 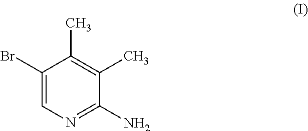
comprising the step of reacting a compound of Formula (IV): 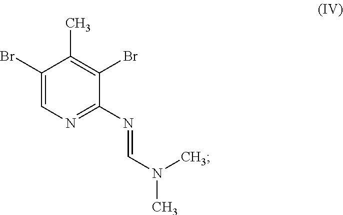
with a methyl zinc compound or methyl zinc salt in the presence of a nickel catalyst to provide said compound of Formula (I).

6. The process according to claim 5 wherein said nickel catalyst is NiCl₂ with a ligand selected from 1,1-bis(diphenylphosphino)methane, (2,2′-bis(diphenylphosphino)-1,1′-binaphthyl), 1,3-bis(dicyclohexylphosphino)propane, triphenylphosphine, 1,2-bis(diphenylphosphino)benzene, 1,2-bis(dicyclohexylphosphino)ethane, and 1,1′-bis(diisopropylphosphino)ferrocene.

7. The process according to claim 5 wherein said nickel catalyst is bis(diphenylphosphino) propane) nickel chloride.

8. The process according to claim 5 wherein said methyl zinc compound or methyl zinc salt is dimethylzinc or a combination of anhydrous zinc bromide and Grignard reagent.

9. A process for preparing a compound of Formula (IV): 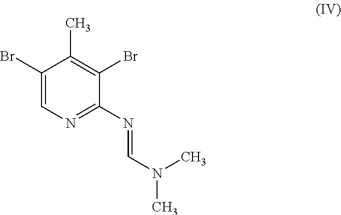
comprising the step of reacting a compound of Formula (II) and a compound of Formula (III): 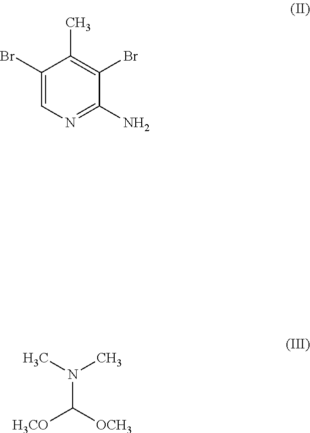
to provide said compound of Formula (IV).

10. A compound having the structure of Formula (IV):

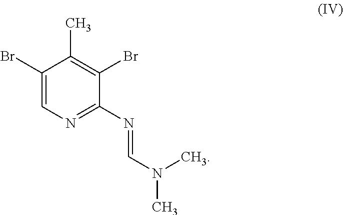

11. A process for preparing a compound of Formula (VI): 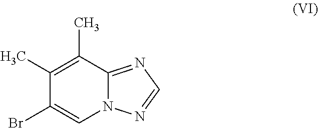
comprising the steps of:
(i) reacting a compound of Formula (IV): 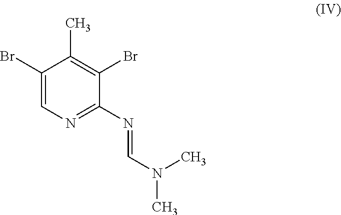
with a methyl zinc compound or methyl zinc salt in the presence of a nickel catalyst to provide said compound of Formula (V): 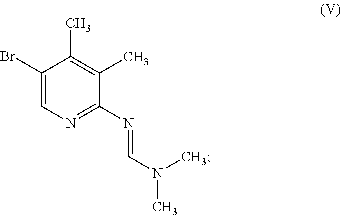
and
(ii) reacting said compound of Formula (V) with hydroxylamine-O-sulfonic acid and 0.5 to 1.3 equivalents of dimethylformamide-dimethylacetal to provide said compound of Formula (VI).

12. A process for preparing a compound of Formula (VI): 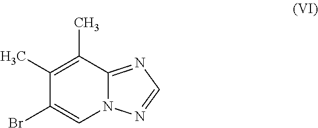
comprising the step of reacting a compound of Formula (V); 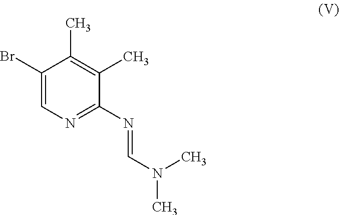
with hydroxylamine-O-sulfonic acid and 0.5 to 1.3 equivalents of dimethylformamide-dimethylacetal to provide said compound of Formula (VI).

13. A process for preparing a compound of Formula (V): 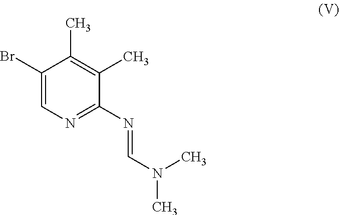
comprising the step of reacting a compound of Formula (IV): 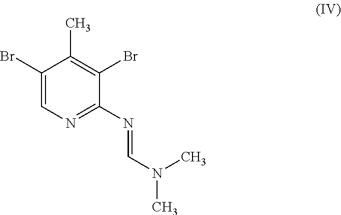
with a methyl zinc compound or methyl zinc salt in the presence of a nickel catalyst to provide said compound of Formula (V).
